(19) United States
(12) Patent Application Publication
(10) Pub. No.: US 2012/0031401 A1
(43) Pub. Date: Feb. 9, 2012

(54) LIGHT BLOCKING CONTAINER WITH VIEWING WINDOW FOR PHOTOSENSITIVE COMPOUNDS

(75) Inventors: Robert L. Berger, Bound Brook, NJ (US); Michael L. Turini, Cranford, NJ (US)

(73) Assignee: SCHERING CORPORATION

(21) Appl. No.: 13/127,408

(22) PCT Filed: Nov. 3, 2009
(86) PCT No.: PCT/US09/63084

§ 371 Date: Oct. 11, 2011

(30) Foreign Application Priority Data
Nov. 3, 2008 (US) 61110909

(51) Int. Cl. A61M 16/10; B65D 85/00

(52) U.S. Cl. 128/203.12; 206/524.1

(57) ABSTRACT

Several embodiments of the present invention provide a container (10) for housing a photosensitive APA compound therein, the container including an outer casing (12), an inner casing (18) disposed within the interior of the outer casing (12), the inner casing (18) configured to house the photo-sensitive APA compound therein, and a viewing window (14) formed through the outer casing (12) to permit viewing of the inner casing (18) from the outside of the outer casing (12), the viewing window (14) including a light-protecting material.

### FIELD OF THE INVENTION

The present invention relates to containers for housing photosensitive materials, such as active pharmaceutical agents (APAs) and the like.

### BACKGROUND

Certain compositions that contain active pharmaceutical agents (APAs) are known to be particularly photosensitive. In particular, certain APA compositions may experience lessened efficacy, or even a complete loss of efficacy, after exposure to ultraviolet rays from light. In some cases, even a very brief exposure to such ultraviolet rays can result in a decreased efficacy of the APA. Thus, it is important for some APAs to be housed in a container that shields the APA from exposure to light.

At the same time, however, the user of the APA often desires to see the contents of the container; so that the user may be aware of how much APA is left, and whether there is enough of the APA to administer a dosage of the APA. Generally, photosensitive APAs are housed in either colored glass bottles, glass bottles that have been treated with a UV-protectant, or in opaque plastic bottles. When the APA is contained in the colored or UV-treated glass bottle, the user may view the contents therein, however, with some difficulty. However, opaque bottles do not offer the ability to see the contents held within. Further, light-protecting materials, including colorants such as carbon black and UV-protectants, which are typically used on glass bottles cannot be used on plastic bottles, since these additives have a tendency to leach from the plastic material and contact the APA contained therein. Many APAs become contaminated when exposed to these additives, rendering the APA useless and potentially harmful to the user.

It is desirable to provide a container which allows the user to view the contents of photosensitive APAs contained therein, and that avoids the problem of APA contamination while simultaneously protecting the photosensitive APAs from exposure to light.

### SUMMARY OF THE INVENTION

In several embodiments, the invention provides a container for housing a photosensitive compound therein, the container including an outer casing, an inner casing disposed within the interior of the outer casing, the inner easing configured to house the photosensitive compound therein, and a viewing window formed through the outer casing to permit viewing of the inner casing from the outside of the outer casing, the viewing window terminating outside of the inner casing, the viewing window being spaced from, and out of contact with, the photosensitive composition, and the viewing window including a light-protecting material.

Other embodiments provide a container for housing at least one photosensitive active pharmaceutical agent therein, the container comprising an outer casing; an inner casing disposed within the interior of the outer casing, the inner casing configured to house the at least one photosensitive active pharmaceutical agent therein; and a viewing window formed through the outer casing to permit viewing of the inner casing from the outside of the outer casing, the viewing window terminating outside of the inner casing, the viewing window being spaced from, and out of contact with, the at least one photosensitive active pharmaceutical agent, and the viewing window comprising a light-protecting material. The outer or inner casing may be a glass or plastic material and may be opaque. The outer casing can include titanium. The outer or inner casing may be comprised of at least one material selected from the group consisting of high density polyethylene, polypropylene, polyester, cyclic olefin copolymer and combinations thereof. The inner casing comprises a plastic material. In certain embodiments the inner easing is substantially transparent.

Suitable materials for the window include polypropylene, cyclic olefin copolymer and combinations thereof The light-protecting material may include a colorant, a UV-protectant and combinations thereof. The container may include a spraying mechanism. The inner and outer casings may be formed of different materials. The container may include, at least one additional layer between the outer and inner casings. The container may include a middle layer comprising a contrast enhancing agent and the agent may be a dark colorant.

Further embodiments provide for a device for dispensing doses of at least one photosensitive active pharmaceutical agent into a nasal cavity of a user, the device comprising a container and a dispenser in communication with the interior of the container, the dispenser configured to urge doses of the at least one photosensitive active pharmaceutical agent from the interior and into the nasal cavity of the user.

Still further embodiments provide for a drug product comprising a container and a photosensitive active pharmaceutical agent therein; wherein the container comprises an outer casing; an inner casing disposed within the interior of the outer casing, the inner easing configured to house the at least one photosensitive active pharmaceutical agent therein; and a viewing window formed through the outer casing to permit viewing of the inner easing from the outside of the outer casing, the viewing window terminating outside of the inner casing, the viewing window being spaced from, and out of contact with, the at least one photosensitive active pharmaceutical agent, and the viewing window comprising a light-protecting material. The drug product may contain at least one photosensitive active pharmaceutical agent selected from the group consisting of mometasone furoate monohydrate, azelastine, oxymetazoline, fluticasone furoate, fluticasone propionate and combinations thereof. The composition may include at least one active pharmaceutical agent, such as mornetasone furoate, such as mometasone furoate monohydrate or mometasone furoate anhydrous, montelukast, oxymetazoline, azelastine, fluticasone furoate, fluticasone propionate and combinations or pharmaceutically acceptable salts thereof.

Still other embodiments provide for a drug product comprising a container and at least one active pharmaceutical agent therein; wherein the container comprises an outer easing; an inner casing disposed within the interior of the outer casing, the inner casing configured to house the at least one photosensitive active pharmaceutical agent therein; and a viewing window formed through the outer easing to permit viewing of the inner casing from the outside of the outer easing, the viewing window terminating outside of the inner casing, the viewing window being spaced from, and out of contact with, the at least one active pharmaceutical agent, and the viewing window comprising a light-protecting material.

### BRIEF DESCRIPTION OF THE DRAWINGS

FIG. 1 is a perspective view of a first embodiment of the subject invention incorporating a view window.

FIG. 2 is a schematic view of the first embodiment of the subject invention incorporating a view window.

FIG. 3A depicts an embodiment of the subject invention incorporating a viewing window.

FIG. 3B is a cross-sectional view of the container wall including the viewing window therein.

FIG. 4A is a cross-sectional view multi-layered container wall formed in accordance with the subject invention.

FIG. 4B is a cross-sectional view of a multi-layered container wall formed in accordance with the subject invention including a viewing window therein.

FIG. 5 is a side view of a container formed in accordance with another embodiment of the subject invention.

FIG. 6 is a cross-sectional view of a container of FIG. 5.

### DETAILED DESCRIPTION

With reference to the Figures, several embodiments of the present invention are directed to a container, which may be used to house liquids, including chemical compounds, and in particular APA-containing compounds (e.g., pharmaceutically-active compounds). Any APA-containing compound or composition may be housed in the container, and in particular, the APA-containing compound may include photosensitive APAs. The term “photosensitive” may include any composition that reacts or degrades in the presence of light, and in particular may include compounds that react in the presence of ultraviolet rays. The term “photosensitive” is not limited to compounds that become completely ineffective in the presence of light, and may include compounds that degrade into undesirable degradants, only slightly react and/or lose effectiveness after exposure to light.

Non-limiting examples of photosensitive compounds include light sensitive APAs, such as mometasone furoate monohydrate, which is sold under the trademark NASONEX® and is sold by Schering Corporation of Kenilworth, N.J. Another example of a nasal spray photosensitive compound is oxymetazoline, which is sold under the trademark AFRIN® by Schering Corporation of Kenilworth, N.J. Another example of a nasal spray photosensitive compound is fluticasone furoate, which is sold under the trademark VERAMYST® by GlaxoSmithKline. Suitable at least one photosensitive active pharmaceutical agent may include mometasone furoate, such as mometasone furoate monohydrate or mornetasone furoate anhydrous, montelukast, oxymetazoline, azelastine, fluticasone furoate, fluticasone propionate and combinations or pharmaceutically acceptable salts thereof.

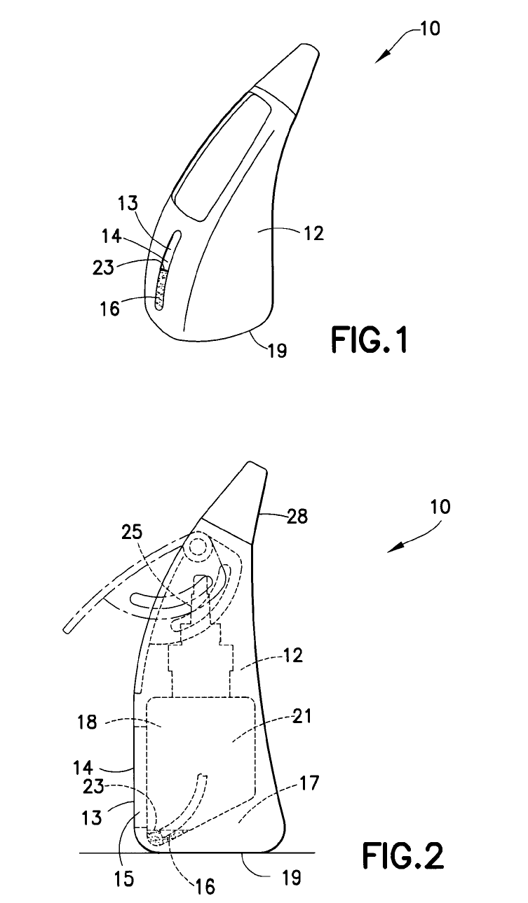

FIG. 1 depicts a first embodiment of a container 10. The container 10 may be used to simply store the photosensitive compound, or it may be used in conjunction with a delivery system. Such delivery systems include droppers, sprayers, injectors, and the like. The container 10 described herein is particularly well-suited to be used conjunction with a nasal dispensing device, however it will be understood that the container 10 may be any type or shape of container, including but not limited to nasal dispensing devices. Nasal dispensers of this type are well known in the art. Examples of such dispensers are shown, for example, in U.S. Pat. Nos. 4,274,560; 4,944,429; and 5,433,343; the entire disclosures of which are incorporated herein by reference.

The container 10 is a dual-container device having an outer casing 12 and an inner casing 18. The outer easing 12 may be made of any material desired, such as a light-protecting material. As used herein, the term “light-protecting material” refers to any material that may be used to shield from exposure to light. For example, light-protecting materials may include opaque materials, through which light may not enter. Light-protecting materials may include colorants or UV-protectants, which allow the user to see through the material, but block light and/or UV rays from passing through the material. For example, the outer casing 12 is opaque, through which transmission of light is inhibited. In one embodiment, the outer casing 12 includes at least one light-protecting material, either attached to one or more surfaces of the outer casing 12 or formed integrally as a part of the outer casing 12. The outer casing 12 may be made from plastic, but it may be made from any desired materials, including glass or other polymeric materials. Suitable materials for the outer casing 12 include high density polyethylene (HDPE), polypropylene, polyester (such as PET, PETg), COC, and other similar materials In general, it is desirable to use a material that provides a good vapor harrier, while maintaining acceptable clarity levels and is not brittle. As will be described in more detail below, the outer easing 12 does not physically come into contact with any photosensitive composition 16 contained in the container 10. Thus, there is no potential for contamination of the photosensitive composition 16 through use of light-protecting materials on the outer casing 12.

The outer casing 12 may include indicia or other markings identifying the materials contained therein if desired. In several embodiments, the outer casing 12 may be of an opaque material. A viewing window 14 may be formed through the outer easing 12. The viewing window 14 may either be formed with the outer casing 12 or may be a separate piece and fitted into the outer casing 12. The viewing window 14 may be made of any material suitable for viewing, and in one embodiment the viewing window 14 is made of plastic. For example, the viewing window 14 may be made from similar materials as the outer easing 12, but may be made of the same or of different materials. For example, the viewing window 14 may be made from monolayer or multi-layered cyclic olefin copolymer (COC), HDPE, COC, polypropylene, or combinations thereof In one embodiment, the viewing window 14 is a multi-layered configuration, which includes more than one layer of material. In other embodiments, the view window may be made from COC with an over-molded PET layer. The lag window 14 is sufficiently transparent to allow viewing therethrough, and desirably includes a light-protecting material for protecting the contents of the outer casing 12 from exposure to light and/or UV rays. The viewing window 14 may be substantially transparent, and the light-protecting material may be a colorant or a UV-protectant.

In one particular embodiment, the container 10 may include an outer casing made from three layers of HDPE and a viewing window 14 made from COC. In this embodiment, the materials used may be bonded together naturally. In another embodiment, the container 10 may include a clear outer casing 12 which is covered by an opaque label, the label having an open portion forming the viewing window 14. In other embodiments, the container 10 may include an outer casing 12 having a plurality of layers of HDPE with a clarified polypropylene stripe forming the viewing window 14.

The light-protecting material may be in the viewing window 14 or it may be applied to one or more outer surfaces 13, 15 of the viewing window 14. For example, the light-protecting material may be formed integrally with the viewing window 14 during formation, or the light-protecting material may be in the form of a sticker, a coating, or the like, which is then applied to one or more of the surfaces 13, 15 of the viewing window 14. The viewing window 14 may be disposed on any desired surface of the outer casing 12. In some embodiments, more than one of the viewing windows 14 may be formed through the outer casing 12.

As will be understood, the outer surface 13 of the viewing window 14 is exposed to the outside of the container 10, while the inner surface 15 of the viewing window 14 is exposed to an interior 17 of the outer casing 12. With the viewing window 14 being sufficiently transparent, the user is capable of seeing through at least a portion of the outer casing 12 into the interior 17 thereof. The viewing window 14 may be any shape or design, including linear, circular, box shaped, curved, irregular, or any other desired shape which will allow viewing through the viewing window 14. Desirably, the viewing window 14 is located at a position close to the bottom 19 of the container 10, so that the user will be able to view low levels of any contents contained in the container 10.

With reference to FIG. 2, the inner casing 18 may be completely contained in the interior 17 of the outer casing 12, although portions of the inner casing 18 may extend beyond the interior 17 of the outer casing 12 if desired. In various embodiments, the inner casing 18 is wholly shielded from exposure to light and/or UV rays by the outer easing 12.

As explained above, the outer casing 12 is made of opaque and/or light-protecting material (including but not limited to colorants and UV-protectants). In addition, the viewing window 14 includes light-protecting materials. As such, the materials contained within the interior 17 of the outer casing 12 will be generally protected from light exposure. The inner casing 18 may thus be made from any desired material, which need not necessarily be light-protected. In some embodiments, the inner casing 18 may be made from plastic or glass. The inner casing 18 may be made from the same material or different material as the outer casing 12,

In several embodiments, the inner casing 18 is at least substantially transparent, manufactured from a non-light protecting material, and may be completely transparent. The inner casing 18 has an interior 21, which houses the photosensitive composition 16. As such, it is preferred that the inner casing 18 be compatible with the photosensitive composition 16 and not include any materials that may contaminate the photosensitive composition 16, including but not limited to colorants and UV-protectant materials. The outer casing 12 and the inner casing 18 may be formed of different materials. With the inner casing 12 and the outer casing 18 being of different materials, the casings 12, 18 may be formed to suit different purposes. The outer casing 12, for example, may be provided with light-protecting materials, while the inner easing 18 may be formed to be compatible with the photosensitive composition 16. In several embodiments, the viewing window 14 terminates at a location outside of the inner casing 18, such that the viewing window 14 is spaced from, and out of contact with, the photosensitive, composition 16 contained in the inner casing 18. Issues of leaching associated with typical light-protecting materials may thus be avoided with the subject invention.

The inner casing 18 may include an access opening 25, which provides access into the interior 21 of the inner casing 18. The access opening 25 is normally covered, such that the photosensitive composition 16 cannot be removed unless the user physically manipulates the container 10 to remove the photosensitive composition. 16. In one embodiment, a dispensing (e.g., nasal dispenser) or dropping mechanism as explained above may be fixed to the container 10 so as to have access to the interior 21 via the access opening 25. In this configuration, there may be a nasal dispenser 28 in communication with the interior 21 of the inner casing 18. The nasal dispenser 28 may be configured to urge doses of the photosensitive compound 16 from the interior 21 and into the nasal cavity of the user. The nasal dispenser 28 and the container 10 collectively define a device for dispensing doses of liquid into a user's nasal cavity. In other embodiments, the inner casing 18 and/or outer casing 12 may include a lid or other mechanism to allow the user to seal off the interior 21 (e.g., seal off the access opening 25) of the inner casing 18.

With the subject invention, the photosensitive composition 16 is contained within the interior 21 of the inner casing 18, and is thus protected from contamination by colorants or UV-protectant materials contained in the outer casing 12 and/or the viewing window 14. The inner casing 18 is contained within the interior 17 of the light-protecting outer casing 12. As such, the photosensitive composition 16 is protected from exposure to light.

The viewing window 14 may extend upwardly from the bottom 19 of the container 10. The viewing window 14 may have sufficient length to allow the top surface 23 of the photosensitive composition 16 to be viewed during normal use. With the ability to view the top surface 23 of the photosensitive composition 16, the amount of the photosensitive composition 16 may be evaluated. Since the outer casing 12 includes the light-protecting viewing window 14, the user may view the contents of the inner casing 18 without having to open the container 10 and expose the photosensitive composition 16 to light. The photosensitive composition 16 is also protected during storage and between uses from light. As such, the photosensitive composition 16 is protected from not only the harmful exposure to light but also from potential contamination from light-protecting materials, such as colorants and UV-protectants.

The interior 21 may be formed to extend below the viewing window 14. In this manner, the photosensitive composition 16 may be contained in the interior 17 at such a low level as to not be viewable through the viewing window 14. This arrangement allows for a slight excess amount of the photosensitive composition 16 to be provided to ensure proper and complete full dosing. With a user believing that there has been full depletion of the photosensitive compound 16. The excess amount allows for variation in dosing and/or assists in maintaining performance of any dispenser and/or assists in allowing for full dosages to be delivered through any dispenser intake (such as a drop tube).

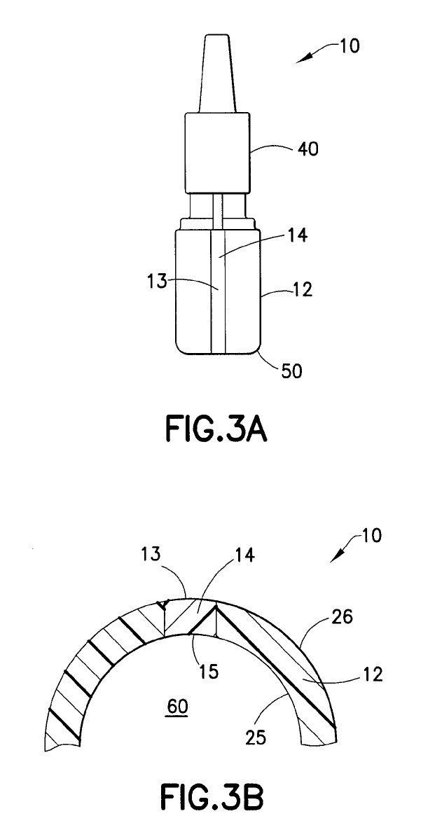

With reference to FIG. 3A, the container 10 is depicted, with an outer casing 12 having a view window 14 that extends from the very bottom region 50 of the container 10 to the upper region 40 of the container 10. FIG. 3B shows a cross-sectional view of the, outer casing 12, including the viewing window 14 therein. As can be seen, the outer casing 12 includes an inner surface 25 and an outer surface 26. Likewise, the viewing window 14 includes an inner surface 15 and an outer surface 13. In the dual-casing embodiment described herein, the inner casing 18 will be disposed in the interior 60 of the outer casing 12. In this fashion, the inner surface 15 of the viewing window 14 and/or the inner surface 25 of the outer casing 12 may include one or more colorants or UV protectant materials without potential for harm of the composition 16 housed in the container 10.

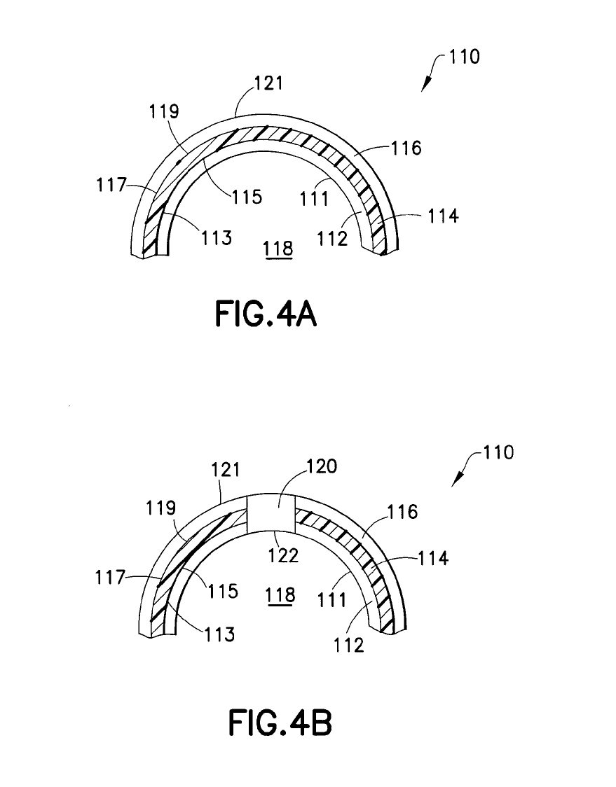

The inner casing 12 and/or the outer casing 18 may be of a multi-layered design. As seen in FIGS. 4A and 4B, the casing 110 (which may be the inner casing 12 and/or the outer casing 18) may include multiple layers. The casing 110 may include, for example, a first layer 112, a second layer 114, and optionally a third layer 116. More than three layers may be incorporated if desired. The inner surface 112 includes an inner layer 111 and an outer layer 113. Similarly, the second layer 114 includes an inner surface 115 and an outer surface 117. The inner surface 115 of the second layer 114 is in contact with the outer surface 113 of the first layer 112. As seen in FIG. 4B, the multi-walled design may include a viewing window 120 disposed therein.

In the multi-layered wall design, the second layer 114 (or any other layers disposed on the outer surface 117 thereof) includes a colorant, UV protectant, or other light-protecting material therein, in this fashion, the colorant, UV protectant or other light-protecting material is further removed from an interior region 118 of the container 110. This further serves to reduce the likelihood that the drug-containing composition housed therein will be exposed to the potentially harmful light-protecting materials incorporated into the container 110.

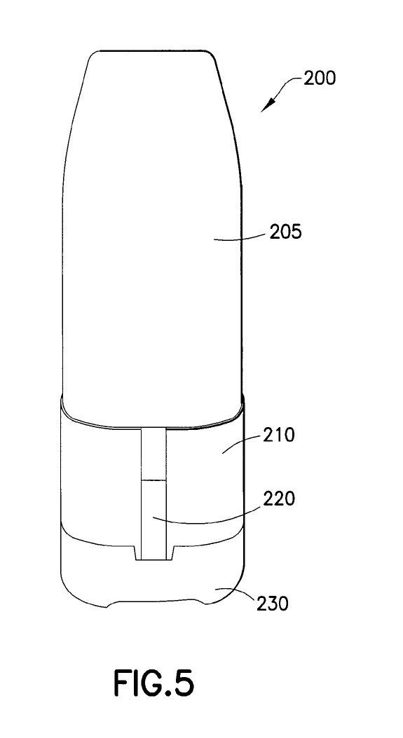

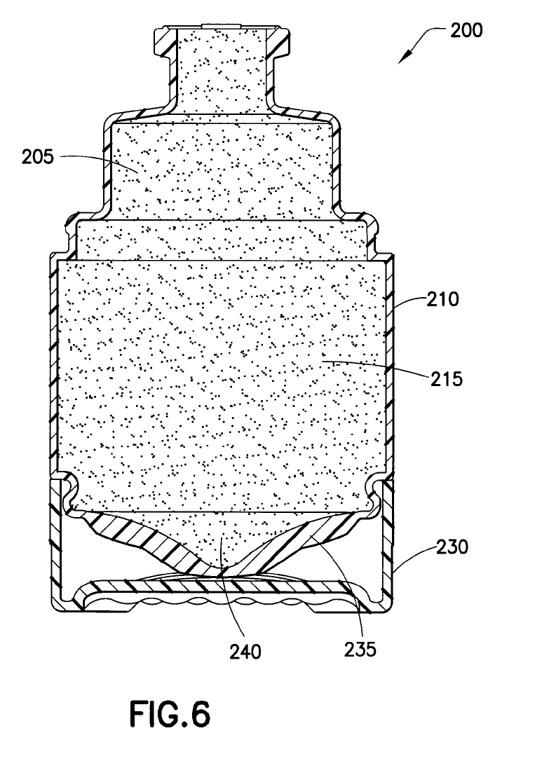

In another embodiment, there may be provided an interior container having a tapered bottom portion to reduce the amount of drug overage. As depicted in FIGS. 5 and 6, there is provided a container 200, having an interior 215, designed for housing a composition containing at least one APA 240. In several embodiments, the composition 240 is liquid in form, but other forms are contemplated such as powders. The container 200 includes a main body portion 210 and a base portion 230. The main body portion 210 and the base portion 230 may be made of the same materials or may be made of different materials, which may include any material compatible with the composition 240 housed therein. In several embodiments, the main body portion 210 and the base portion 230 are made of polymeric materials, such as HDPE, COC, PET, polypropylene and combinations thereof. The main body portion 210 and the base portion 230 may be colored or they may be clear. In one embodiment, the main body portion 210 and the base portion 230 are a color that is in contrast to the color of the composition housed therein, and may be opaque. As with the containers described above, the container 200 may include indicia on its body indicating the contents housed therein.

The main body portion 210 and the base portion 230 may be formed together as a single-piece molded unit, or they may be formed as separately molded pieces and fitted together. As will be appreciated by one of skill in the art, in view of the components of each portion of the container 200, forming the main body portion 210 and the base portion 230 as separate pieces may offer significant manufacturing advantages. The main body portion 210 and the base portion 230 may be attached together by any known means, including by ultrasonic welding, mechanical attachment, adhesives, or other desired means. When fitted together, the main body portion 210 and the base portion 230 form an integrally connected container 200, which is fluid-tight to safely and effectively house the composition 240 housed therein.

The main body portion 210 includes a visible viewing window 220, which allows the user to view the contents in the interior 215 of the container 200. In a several embodiments, the viewing window 220 is in the form of a vertical stripe, however other geometric configurations are contemplated, including ovals, blocks, and other desired figures. The viewing window 220 may be disposed on any side of the main body portion 210, including the front, back, or side. There may be more than one viewing windows 220 disposed on the main body portion 210 if desired. It is desired that the viewing window 220 be of a relatively thin size, to allow viewing of the contents in the container 200, white allowing the main body portion 210 to form the majority of the container 200 body. In one embodiment, the thickness of the viewing window 220 is less than approximately 5% of the circumference of the container 200 body. The viewing window 220 may have any desired length, and in one embodiment, the viewing window begins at or near the location where the base portion 230 is joined with the main body portion 210. The viewing window 220 may be of a sufficient length that it extends at or near the top of the main body portion 210. In one embodiment, the container 200 body includes a label or sticker applied to the outer surface, the label being sufficiently opaque or contrasting colored and further including a viewing window 220 as part of the label.

In one particular embodiment, as can be seen in FIG. 6, the base portion 230 includes a tapered bottom 235. The tapered bottom 235 may extend towards the interior 215 as much as desired. The tapered bottom 235 may be positioned to extend approximately to the area where the base portion 230 and the main body portion 210 are joined. The composition 240 is housed in the interior 215 of the container 200, and is further contained in the area formed by the tapered bottom 235 of the base portion 230.

The tapered bottom 235 of the base portion 230 is useful in reducing the amount of overage of composition 240 that may be left in the container 200 after the user believes that the container 200 is empty. This configuration is especially useful when a delivery system (not shown) is attached to the container 200, and is particularly useful when the delivery system includes a spray attachment. In typical spray bottle configurations, the delivery system includes a hose that extends into the container. The hose typically is unable to effectively remove the composition that rests at the very bottom of the container. With a typical configuration, the bottom is flat, which leaves a large region for excess composition to remain. In some instances, it has been found that there may be enough composition left to administer as many as 8-10 dosages remaining unused in such typical containers.

In contrast, with the tapered bottom 235 of the present invention, the base region 230 provides much less area in the interior 215 of the container 200, and thus greatly reduces the amount of composition 240 remaining in the container 200 after use. In fact, it has been determined that there is typically remaining enough of the composition 240 to provide about 2-4 dosages unused in the container 200. The invention provided herein thus reduces the wasted amount of composition 240 to about 25% of the prior art devices. in addition, the viewing window 220 may be configured to extend only to the top of the tapered bottom 235, so the user is not aware that the small amount of composition 240 remains, and thus will not attempt to provide a dosage when the level of composition 240 is not sufficiently high to reach the delivery system.

The descriptions of the embodiments of the invention have been presented for purpose of illustration and description. They are not intended to be exhaustive or to limit the invention to the precise forms disclosed, and obviously many modifications and variations are possible in light of the above teaching.

What is claimed is:

1. A container for housing at least one photosensitive active pharmaceutical agent therein, the container comprising:
a. an outer casing;
b. an inner casing disposed within the interior of the outer casing, the inner casing configured to house the at least one photosensitive active pharmaceutical agent therein; and
c. a viewing window formed through the outer casing to permit viewing of the inner casing from the outside of the outer casing, the viewing window terminating outside of the inner casing, the viewing window being spaced from, and out of contact with, the at least one photosensitive active pharmaceutical agent, and the viewing window comprising a light-protecting material.

2. The container of claim 1, wherein the outer casing comprises a plastic material.

3. The container of claim 1, wherein the outer casing is opaque.

4. The container of claim 1, wherein the outer casing comprises titanium.

5. The container of claim 1, wherein the outer casing comprises at least one material selected from the group consisting of high density polyethylene, polypropylene, polyester, cyclic olefin copolymer and combinations thereof.

6. The container of claim 1, wherein the inner casing comprises a plastic material.

7. The container of claim 1, wherein the inner casing is substantially transparent.

8. The container of claim 1, wherein the inner casing comprises a material selected from the group consisting of high density polyethylene, polypropylene, polyester, cyclic olefin copolymer and combinations thereof.

9. The container of claim 1, wherein the window comprises a material selected from the group consisting of polypropylene, cyclic olefin copolymer and combinations thereof.

10. The container of claim 1, wherein the window is a cyclic olefin copolymer.

11. The container of claim 1, wherein the light-protecting material comprises a colorant.

12. The container of claim 1, wherein the light-protecting material comprises a UV-protectant.

13. The container of claim 1, comprising a spraying mechanism.

14. The container of claim 1, wherein the inner and outer casings are formed of different materials.

15. The container of claim 1, further comprising at least one additional layer between the outer and inner casings.

16. The container of claim 1, further comprising a middle layer comprising a contrast enhancing agent.

17. The container of claim 16, wherein the contrast enhancing agent is a dark colorant.

18. A device for dispensing doses of at least one photosensitive pharmaceutical agent into a nasal cavity of a user, the device comprising:
a. a container as set forth in claim 1; and
b. a dispenser in communication with the interior of the container, the dispenser configured to urge doses of the at least one photosensitive active pharmaceutical agent from the interior and into the nasal cavity of the user.

19. A device as in claim 18, wherein the at least one photosensitive active pharmaceutical agent comprises mometasone furoate monohydrate.

20. A drug product comprising a container and at least one photosensitive active pharmaceutical agent therein; wherein the container comprising:
a. an outer casing;
b. an inner casing disposed within the interior of the outer casing, the inner casing configured to house the at least one photosensitive active pharmaceutical agent therein; and
c. a viewing window formed through the outer casing to permit viewing of the inner casing from the outside of the outer casing, the viewing window terminating outside of the inner casing, the viewing window being spaced from, and out of contact with, the at least one photosensitive active pharmaceutical agent, and the viewing window comprising a light-protecting material.

21. The drug product of claim 20, wherein the at least one photosensitive active pharmaceutical agent is selected from the group consisting of mometasone furoate, mometasone furoate monohydrate, oxymetazoline, azelastine, montelukast, fluticasone furoate, fluticasone propionate and combinations and pharmaceutically acceptable salts thereof.
